(19) United States
(12) Patent Application Publication
(10) Pub. No.: US 2025/0186393 A1
(43) Pub. Date: Jun. 12, 2025

(54) TREATMENT OF BREAST CANCER WITH AMCENESTRANT

(71) Applicant: SANOFI, Paris (FR)

(72) Inventors: Patrick COHEN, Paris (FR); Elisabeth DE KERMADEC, Cambridge, MA (US); Qianying LIU, Cambridge, MA (US); Gautier PAUX, Cambridge, MA (US)

(73) Assignee: SANOFI, Paris, FR (FR)

(21) Appl. No.: 18/846,183

(22) PCT Filed: Mar. 13, 2023
(86) PCT No.: PCT/IB2023/052408

§ 371 Date: Sep. 11, 2024

Related U.S. Application Data
(60) Provisional application No. 63/319,373, filed on Mar. 13, 2022.
(60) Provisional application No. 63/375,108, filed on Sep. 9, 2022.

(30) Foreign Application Priority Data
Dec. 9, 2022 (EP) 22315323.0

(51) Int. Cl. A61K 31/40; A61P 35/00

(52) U.S. Cl. CPC A61K 31/40; A61P 35/00

(57) ABSTRACT

The present disclosure provides methods of treating breast cancer with amcenestrant.

### CROSS-REFERENCE TO RELATED APPLICATIONS

This application claims priority from U.S. App. No. 63/319,373, filed on Mar. 13, 2022; U.S. App. No. 63/375,108, filed on Sep. 9, 2022; and EP App. No. 22315323.0, filed on Dec. 9, 2022. The disclosures of the aforementioned priority applications are incorporated herein by reference in their entirety.

### BACKGROUND

Breast cancer is the most commonly diagnosed cancer and the second leading cause of death in women in the United States. Both endogenous and exogenous steroid hormones such as estrogen and progesterone have been implicated in the pathogenesis of breast cancer. Clinical treatment decisions are driven in part by the expression status of hormone receptors (HRs), more specifically estrogen receptors (ERs) and progesterone receptors (PgRs), and of human epidermal growth factor receptor 2 (HER2). There are three molecular subtypes of breast cancer: HER2+, HR+/HER2−, and ER−/PgR−/HER2− (triple negative).

A key protein target in the treatment of breast cancer is estrogen receptor alpha (ERα), a hormone regulator transcription factor that is encoded by the ESR1 gene and expressed at elevated levels in approximately about 75% of breast tumors. ERα enables breast tumors to respond to the mitogenic actions of estrogens. ER-positive breast cancers respond well to therapy targeting ER signaling either through competitive binding of ER by antagonists, such as tamoxifen, or by blocking the production of estrogen by aromatase inhibitors (AIs).

Selective ER degraders (SERDs) are competitive ER antagonists that also induce conformational changes that lead to the degradation of ER via a ubiquitin-proteasome system. The unique dual function of SERDs (ER antagonism and depletion) may enable them to block ER signaling in cellular settings where other endocrine agents, such as tamoxifen or AIs, have failed.

Fulvestrant is currently the only approved SERD medicine available on the market. However, this therapy is limited by its poor pharmaceutical properties, which necessitate intramuscular administration and limit the applied dose, exposure, and receptor engagement.

Amcenestrant is a potent, orally bioavailable, and selective estrogen receptor (ER) inhibitor that also belongs to the SERD class of compounds. Amcenestrant antagonizes the binding of estradiol to ER and promotes the transition of ER to an inactive conformation that leads to up to 98% receptor degradation at nanomolar concentrations in cellular assays.

Sequential hormonal therapy (alone or in combination) is currently the standard of care in the metastatic breast cancer setting for ER+/HER2− patients without rapidly progressing visceral or symptomatic metastases. Unfortunately, not all patients respond to first-line hormonal therapy as they present with primary or de novo resistance, and some patients who initially respond subsequently have breast cancer progression (acquired resistance). Resistance to endocrine therapies is frequent, but relapsed tumors remain dependent on ER, which is highlighted by patient responses to second- and third-line endocrine therapies after failure of an earlier line of hormonal therapy. In locally advanced/metastatic breast cancer patients who progress after one or more lines of hormonal therapy, the current choice of further single agent treatment is mainly represented by three classes of compounds: the selective estrogen receptor modulator (SERM) tamoxifen; AIs such as letrozole, anastrozole, and exemestane; and the SERD fulvestrant.

Overall, in metastatic breast cancer patients who progressed after one or two lines of hormonal based therapy and who received a further hormonal single agent therapy, the efficacy results indicate a Progression-Free Survival (PFS) of 1.9 to 5.1 months across studies.

Thus, there remains a need for an effective and well-tolerated SERD therapy with improved route of administration (oral versus intramuscular route), bioavailability, and maintenance of ER receptor blockade.

### SUMMARY

The present disclosure provides a method of treating breast cancer, comprising orally administering to a patient in need thereof a 400 mg dose of amcenestrant or a pharmaceutically acceptable salt thereof.

In one aspect, the present disclosure provides a method for reducing disease progression or death, or risk of disease progression or death, as compared to treatment with a therapy selected from fulvestrant, an aromatase inhibitor, and a selective estrogen receptor modulator, in a patient in need thereof, wherein the patient has ER+/HER2− advanced/metastatic breast cancer with mutated estrogen receptor 1 (ESR1), the method comprising administering to the patient amcenestrant or a pharmaceutically acceptable salt thereof.

In one aspect, the present disclosure provides a a method of treating ER+/HER2− advanced/metastatic breast cancer with mutated estrogen receptor 1 (ESR1) in a patient in need thereof, the method comprising administering to the patient amcenestrant or a pharmaceutically acceptable salt thereof.

In some embodiments, the treatment method herein results in a reduction of 10.0% in risk of disease progression or death as compared to treatment with a therapy selected from fulvestrant, an aromatase inhibitor, and a selective estrogen receptor modulator.

In some embodiments, the aromatase inhibitor is exemestane, letrozole, or anastrozole.

In some embodiments, the selective estrogen receptor modulator is tamoxifen.

In some embodiments, the present treatment method increases the progression-free survival (PFS) of the patient. In some embodiments, the method results in a median PFS of about 3.7 months.

In some embodiments, amcenestrant or a pharmaceutically acceptable salt thereof is administered to the patient orally at a dose of 400 mg daily, optionally once daily. In some embodiments, amcenestrant or a pharmaceutically acceptable salt thereof is provided as a capsule or a tablet, optionally comprising 100 mg of amcenestrant per capsule or tablet. In some embodiments, amcenestrant or a pharmaceutically acceptable salt thereof is administered to the patient in the morning regardless of food status.

In some embodiments, the breast cancer is advanced breast cancer. In some embodiments, the advanced breast cancer is locally advanced cancer that is not amenable to radiation therapy or surgery in a curative intent. In some embodiments, the breast cancer is metastatic.

In some embodiments, the patient is a pre- or post-menopausal woman, or a man. In some embodiments, the patient is a post-menopausal woman.

In some embodiments, the patient is resistant to endocrine therapy.

In some embodiments, the patient has been previously treated with at least one line, optionally one or two lines, of endocrine therapy for advanced breast cancer, optionally wherein the patient's breast cancer progressed during or after treatment with the previous endocrine therapy. In some embodiments, the patient has been previously treated with adjuvant endocrine therapy and relapsed after the first two years of the adjuvant endocrine therapy or within 12 months of completing the adjuvant endocrine therapy. In some embodiments, the previous adjuvant endocrine therapy is selected from treatment with tamoxifen, fulvestrant, or an aromatase inhibitor, optionally wherein the aromatase inhibitor is exemestane, letrozole, or anastrozole.

In some embodiments, the patient has been previously treated with chemotherapy or targeted therapy. In some embodiments, the patient has been previously treated with no more than one chemotherapy or one targeted therapy for advanced or metastatic disease.

In some embodiments, the patient has been previously treated with a CDK4/6 inhibitor. In some embodiments, the patient has not been previously treated with an mTOR inhibitor and/or with a SERD other than fulvestrant.

In some embodiments, the patient experiences no clinically significant bradycardia, QTc prolongation, or visual disturbances after the treatment.

In another aspect, the present disclosure provides an article of manufacture or kit, comprising amcenestrant and instructions for use for treating ER+/HER2− breast cancer in the present treatment method.

Also provided herein is amcenestrant or a pharmaceutically acceptable salt thereof for use in treating breast cancer in the present treatment method, as well as use of amcenestrant or a pharmaceutically acceptable salt thereof in the manufacture of a medicament for treating breasting cancer in the present treatment method.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a graphical diagram showing the clinical study design. C=Cycle; D=day; IMP=investigational medicinal product; OS=overall survival; PD=progressive disease; R=randomization.

FIG. 2 is a Kaplan-Meier graph of the PFS primary endpoint assessed by ICR in the ITT population.

FIG. 3 is a Kaplan-Meier graph of the OS key secondary endpoint in the ITT population.

FIG. 4 is a forest plot showing subgroups analyses of PFS based on ICR assessment by baseline characteristics in the ITT population.

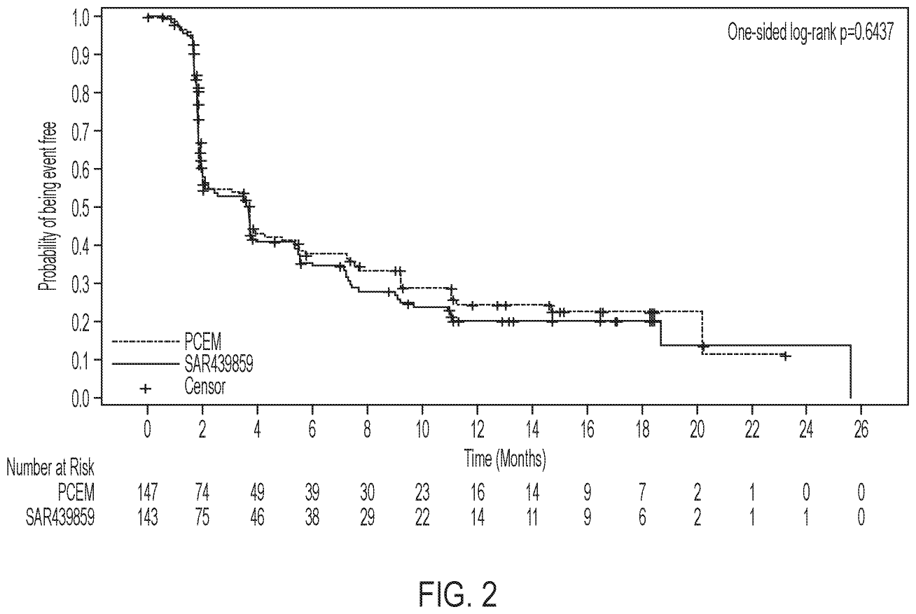

FIGS. 2-4 display results stemming from a trial cut-off date of Feb. 15, 2022 and a patients' database lock dated Mar. 8, 2022.

### DETAILED DESCRIPTION

The present disclosure provides a therapy with amcenestrant for the treatment of breast cancer, including in patients with ESR1-mutation.

It has been discovered that amcenestrant, a SERD, when used at a 400 mg QD, is able to provide a numerically similar progression-free survival compared to endocrine therapy of physician's choice in patients with ER+/HER2− advanced/metastatic breast cancer.

The treatment regimen described herein has been shown to achieve a median PFS of 3.6 months (95% CI 2.0 to 3.9). Hence, the treatment regimen described herein provides a median time of 3.6 months until the patient shows progressive disease or death due to any cause, whichever comes first.

It has also been discovered that amcenestrant, when used at a 400 mg QD, is able to provide a numerically longer progression-free survival compared to endocrine therapy of physician's choice in ER+/HER2− advanced/metastatic breast cancer patients with ESR1-mutation. In said patients, the amcenestrant treatment regimen described herein has been shown to achieve a median PFS of 3.7 months (95% CI 1.9 to 7.2). Hence, the treatment regimen described herein provides a median time of 3.7 months until the patient shows progressive disease or death due to any cause, whichever comes first.

The safety profile of the treatment regimen described herein is further detailed below and is consistent with the one observed in earlier studies.

### I. Amcenestrant

Amcenestrant is a potent, orally bioavailable, and selective ER inhibitor that belongs to the SERD family of compounds. Amcenestrant has complete estrogen receptor antagonist properties and accelerates the proteasomal degradation of ER. Amcenestrant (laboratory code SAR439859) has the chemical name 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or 8-(2,4-dichlorophenyl)-9-(4-{[(3S)-1-(3-fluoropropyl)pyrrolidin-3-yl]oxy}phenyl)-6,7-dihydro-5Hbenzo[7]annulene-3-carboxylic acid (C₃₁H₃₀C₁₂FNO₃). It is described in the patent application WO 2017/140669. Amcenestrant has the following structural formula (I):

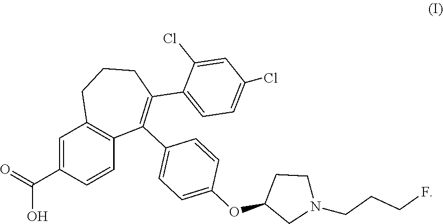 [SMILES: O=C(O)c1ccc2c(c1)CCCC(c1ccc(Cl)cc1Cl)=C2c1ccc(O[C@H]2CCN(CCCF)C2)cc1]

Amcenestrant may be provided as a zwitterion (i.e., a globally neutral molecule having one acidic and one basic group), with no additional counterions. Amcenestrant may also be provided in the form of a salt with one or more additional acidic or basic molecule(s). Unless otherwise noted, the dose of amcenestrant administered to a patient refers to the dose of free zwitterionic (i.e., uncharged) amcenestrant administered and does not include the weight of any counterions.

As used herein, amcenestrant may be provided in a pharmaceutical composition comprising a therapeutically effective amount of amcenestrant or a pharmaceutically acceptable salt thereof, with or without other active ingredients. The pharmaceutical composition may typically be in the form of, e.g., a liquid solution, dispersion, suspension, tablet, capsule, or the like. The pharmaceutical composition may comprise inactive ingredients that are pharmaceutically acceptable excipients and/or carriers. Amcenestrant is typically administered orally (i.e., p.o., PO, or per os). Patients may take the medication with food or without food.

In some embodiments, amcenestrant is suitable for oral administration formulated as a tablet or capsule. In some embodiments, amcenestrant is suitable for oral administration formulated as a capsule comprising 100 mg of amcenestrant.

In some embodiments, amcenestrant or a pharmaceutically acceptable salt thereof is administered to the patient orally at a dose of 400 mg daily. In some embodiments, amcenestrant or a pharmaceutically acceptable salt thereof is administered to the patient once daily.

In some embodiments, amcenestrant or a pharmaceutically acceptable salt thereof is administered to a patient in need thereof.

### II. Treatment Regimens

As used herein, the term “treating” or “treatment” means to arrest, slow down, or reduce disease progression or death, including risk thereof. In some embodiments, treating or treatment means reducing disease progression or death, including risk thereof. In some embodiments, treating or treatment means reducing disease progression or death, including risk thereof, as compared to treatment with a therapy selected from fulvestrant, an aromatase inhibitor, and a selective estrogen receptor modulator, in a patient in need thereof with ER+/HER2− advanced/metastatic breast cancer with mutated estrogen receptor 1 (ESR1).

The patient in need thereof may receive a treatment regimen of the present disclosure until the patient no longer benefits from the treatment, shows disease progression, or shows unacceptable toxicity.

As used herein, administration of amcenestrant includes self-administration by the patient (e.g., oral intake by the patient).

In some embodiments, amcenestrant is administered in a total daily amount of 400 mg (e.g., provided as four capsules, each comprising 100 mg of amcenestrant). Amcenestrant may be administered orally once a day (QD) (i.e., once every 24 hours). In some embodiments, amcenestrant is administered in the morning, at approximately the same time each day (±three hours) regardless of food-status. For the purposes of treatment, a cycle is artificially defined as a 4-week period.

In some embodiments, the treatment is continued until the patient no longer benefits from the treatment. In some embodiments, the treatment continues until the patient shows unacceptable toxicity. In some embodiments, the treatment continues until the patient shows disease progression.

During the treatment period, the patient is monitored regularly for disease status and/or for dose adjustment.

During the treatment period, the patient may advantageously also be monitored for possible side effects, including toxicities and adverse events. In particular, the following are potential side effects and risks anticipated in humans:
Gastrointestinal toxicities, including anorexia, nausea, vomiting, diarrhea (and possibly dehydration and electrolyte imbalance in severe cases), and upper and/or lower abdominal pain.
Changes in liver function, including elevated liver enzymes and bilirubin.
Hematological toxicities potentially presenting with laboratory abnormalities (leukopenia, neutropenia, thrombocytopenia, anemia), infections, and neutropenic fever.
Effects in the female reproductive system (ovary, uterus, cervix, vagina, mammary glands).
Risk of male infertility.
Risk of photosensitivity.
Risk of severe rash.
Theoretical risk of osteoporosis, due to the mechanism of action of amcenestrant, in case of long term exposure.
Risk of drug-drug interaction.

During the treatment period, the patient may be advised to avoid concurrent use of the following therapies/medications:
drugs that are strong inducers of CYP3A;
herbal medications and food supplements including St John's Wort and genistein;
drugs that are sensitive substrates of OATP1B1/1B3 including asunaprevir, atorvastatin, bosentan, danoprevir, fexofenadine, glyburide, nateglinide, pitavastatin, pravastatin, repaglinide, rosuvastatin, and simvastatin acid;
drugs that have UGT inhibition potential and are contraindicated with UGT substrates, including, but not limited to, atazanavir and probenecid.

### III. Selection of Patients

The present therapy may be used to treat breast cancer adult patients (e.g., patients ≥18 years of age). In some embodiments, the patient has an ER+/HER2− breast cancer that is locally advanced and not amenable to radiation therapy or surgery in a curative intent, and/or metastatic breast cancer (also designated hereafter as “locally advanced/metastatic” breast cancer, or “advanced/metastatic” breast cancer). In some embodiments, patients have locally advanced/metastatic breast cancer and have failed (i.e., exhibited disease progression) during or after treatment with one or more other hormonal therapies. In some embodiments, the patient is a postmenopausal woman, a premenopausal woman, or a man. In some embodiments, the patient has ER+/HER2− locally advanced or metastatic breast cancer, and has previously received ≤2 prior lines of ET; and ≤1 prior chemotherapy or ≤1 targeted therapy in the advanced/metastatic setting. In some embodiments, the patient had no experience of prior use of mTOR inhibitors or SERDs other than fulvestrant. In further embodiments, the therapy for such patients comprises oral administration of amcenestrant at a dose of 400 mg daily.

In some embodiments, the patient has a histological or cytological proven diagnosis of adenocarcinoma of the breast. The patient may have evidence of either locally advanced disease not amenable to radiation therapy or surgery in a curative intent, and/or metastatic disease.

A primary tumor or any metastatic site can be considered to be positive for ER if ≥1% of cells are positively stained in a tumor cell staining immunohistochemistry (IHC) assay. The patient may not have a primary tumor that is ER positive and any further metastatic lesion that is ER negative.

A primary tumor or any metastatic site can be judged to be not overexpressing HER2 based on analysis of a tumor sample by IHC (0, 1+), or in situ hybridization-negative based on single-probe average HER2 copy number <6.0 signals/cell or dual-probe HER2/centromeric probe for chromosome 17 (CEP17) ratio <2 with an average HER2 copy number <6.0 signals/cell as per American Society of Clinical Oncology guidelines. The patient may not have a primary tumor that is HER2 negative and any further metastatic lesion that is HER2 positive.

In some embodiments, prior chemotherapy (including antibody drug conjugates) or targeted therapy is allowed. In some embodiments, the patient has received no more than one prior chemotherapeutic or one targeted therapy regimen for advanced/metastatic disease. In some embodiments, the patient has previously received a prior treatment with a CDK4/6 inhibitor. In some embodiments, the patient has undergone prior treatment with a CDK4/6 inhibitor in combination with fulvestrant or an AI.

In some embodiments, the patient has received at least six months of a continuous prior endocrine therapy for advanced breast cancer and has progressed while on endocrine therapy (in single agent or in combination). In some embodiments, the number of prior advanced hormonal lines is two or less. In further embodiments, a patient has experienced a relapse while on adjuvant endocrine therapy but after the first two years, or a relapse within 12 months of completing adjuvant endocrine therapy.

A postmenopausal woman is defined as:
i) a woman >60 years of age; or
ii) a woman <60 years of age:
   with spontaneous cessation of menses >12 months prior to amcenestrant treatment start in the absence of chemotherapy, tamoxifen, and toremifene;
   with cessation of menses of duration ≤12 months or secondary to hysterectomy AND have follicle stimulating hormone (FSH) level in the postmenopausal range according to institutional standards (or >34.4 IU/L if institutional range is not available) prior to amcenestrant treatment start;
   who have received hormonal replacement therapy but have discontinued this treatment AND have FSH level in the postmenopausal range according to institutional standards (or >34.4 IU/L if institutional range is not available) prior to amcenestrant treatment start;
   with status post bilateral surgical oophorectomy; or
   postbilateral ovarian ablation through pelvic radiotherapy.

As used herein, “amcenestrant treatment start” designates the day the treatment with amcenestrant is initiated, or 1, 2, or 3 days earlier.

In some embodiments, the patient is a premenopausal woman (i.e., not a post-menopausal woman) or a man. In some embodiments, male patients with no prior bilateral orchiectomy and pre/perimenopausal women begin treatment with a GnRH agonist at least four weeks prior to treatment.

In some embodiments, the patient does not have an Eastern Cooperative Oncology Group (ECOG) performance status ≥2. The criteria for ECOG status are defined in Table 1 below.

TABLE 1
Eastern Cooperative Oncology Group performance status scale

| Grade | Description |
|---|---|
| 0 | Fully active, able to carry on all pre-disease performance without |
| | restriction |
| 1 | Restricted in physically strenuous activity but ambulatory and |
| | able to carry out work of a light or sedentary nature, e.g., light |
| | housework, office work |
| 2 | Ambulatory and capable of all selfcare but unable to carry out |
| | any work activities. Up and about more than 50% of waking |
| | hours |
| 3 | Capable of only limited selfcare, confined to bed or chair more |
| | than 50% of waking hours |
| 4 | Completely disabled. Cannot carry on any selfcare. Totally |
| | confined to bed or chair |
| 5 | Dead |

In some embodiments, the patient does not have significant concomitant illness, including a psychiatric condition that would adversely affect the patient's adherence to the treatment methods described herein. In further embodiments, the patient does not have a medical history or ongoing gastrointestinal disorders potentially affecting the absorption of an oral medication (e.g., amcenestrant), and is not unable to swallow normally and to take capsules. In some embodiments, the patient has not undergone major surgery within four weeks prior to amcenestrant treatment start.

In some embodiments, the patient does not have any other cancer. The patient may have an adequately treated basal cell or squamous cell skin cancer or in situ cervical cancer or any other cancer from which the patient has been disease free for >3 years.

In some embodiments, the patient does not have an abnormal coagulation profile or any history of coagulopathy within the six months prior to the first dose of amcenestrant, including a history of deep vein thrombosis or pulmonary embolism. The patient may (i) have adequately treated catheter related venous thrombosis that occurred more than one month prior to the first dose of IMP or (ii) be treated with an anticoagulant (e.g., warfarin or heparin) for a thrombotic event that occurred more than six months before enrollment, or for an otherwise stable and allowed medical condition (e.g., well controlled atrial fibrillation), provided that dose and coagulation parameters are stable for at least one month prior to the first dose of amcenestrant.

In some embodiments, the patient does not have known brain metastases that are untreated, symptomatic, or require therapy to control symptoms. A patient with brain metastases may be treated with the present treatment regimen if they (i) have completed treatment (whole brain radiotherapy, radiosurgery, or combination) at least four weeks prior to start of study treatment, (ii) have recovered from the effects of this treatment, and (iii) are neurologically stable. In some embodiments, a patient has discontinued use of corticosteroid for brain metastases without the subsequent appearance of symptoms for ≥2 weeks prior to first administration of amcenestrant.

In some embodiments, the patient does not have lack of improvement of any prior treatment related adverse reaction to <Grade 2, except for alopecia according to the National Cancer Institute Common Terminology Criteria for Adverse Events (NCI CTCAE) v5.0.

In some embodiments, the patient has not undergone prior treatment with mammalian target of rapamycin inhibitors or any other SERD compound, except fulvestrant if stopped for at least three months before amcenestrant treatment start.

In some embodiments, the patient is not treated with drugs that have the potential to inhibit UGT, including, but not limited to, atazanavir and probenecid, for less than two weeks before the start of treatment or five elimination half-lives, whichever is longer. In further embodiments, the patient is not treated with strong CYP3A inducers within two weeks before the start of treatment or five elimination half-lives, whichever is longer. In yet further embodiments, the patient is not treated with drugs that are sensitive substrates of OATP1B1/B3 (e.g., asunaprevir, atorvastatin, bosentan, danoprevir, fexofenadine, glyburide, nateglinide, pitavastatin, pravastatin, repaglinide, rosuvastatin, and simvastatin acid).

In some embodiments, the patient does not have inadequate hematological function including neutrophils <1.5×10⁹/L or platelet count <100×10⁹/L. In further embodiments, the patient does not have a prothrombin time/international normalized ratio (INR)>1.5 times the upper limit of normal (ULN) or outside therapeutic range if receiving anticoagulation that would affect the prothrombin time/INR. In some embodiments, the patient does not have inadequate renal function with serum creatinine ≥1.5×ULN or between 1.0 and 1.5×ULN with glomerular filtration rate <60 mL/min/1.73 m² as estimated using the abbreviated Modification of Diet in Renal Disease formula, shown below:

$$GFR ( mL / min / 1.73 {m}^{2} ) = 175 \times ( Scr ) - 1.154 \times ( Age ) - 0.203 \times ( 0.742 if female ) \times ( 1.212 if African American ) ( conventional units )$$

GFR=glomerular filtration rate; Scr=serum creatinine

In further embodiments, the patient does not have inadequate liver function, indicated by aspartate aminotransferase >3×ULN, ALT>3×ULN, or total bilirubin >1.5×ULN. If the patient has hepatic metastases, AST and ALT<5×ULN are acceptable.

### IV. Treatment Outcomes and Tumor Response Evaluation

The present therapy may result in a complete response (CR), partial response (PR), or stable disease (SD) in patients, and may prevent progressive disease (PD).

In some embodiments, patients have measurable disease at baseline. Measurable disease is defined by the presence of at least one measurable lesion. A measurable lesion refers to a tumor lesion that has been accurately measured in at least one dimension (in the plane of measurement that is to be recorded) with a minimum longest diameter of:
(i) 10 mm by CT scan (CT scan slice thickness no greater than 5 mm);
(ii) 10 mm caliper measurement by clinical exam (lesions which cannot be accurately measured with calipers should be recorded as nonmeasurable); or
(iii) 20 mm by chest X-ray.

A measurable malignant lymph node is a lymph node that is pathologically enlarged and measurable. A malignant lymph node must be ≥15 mm in short axis when assessed by CT scan (CT scan slice thickness recommended to be no greater than 5 mm).

In some embodiments, the various levels of response can be evaluated in accordance with Tables 2 and 3 below.

TABLE 2
Evaluation of Target Lesions

| Response | |
|---|---|
| Classification | Criteria |
| Complete | Disappearance of all target lesions. Any pathological lymph nodes |
| Response (CR) | (whether target or nontarget) must have reduction in short axis to <10 |
| | mm. |
| Partial | At least a 30% decrease in the sum of diameters of target lesions, |
| Response (PR) | taking as reference the baseline sum diameters. |
| Progressive | At least a 20% increase in the sum of diameters of target lesions, taking |
| Disease (PD) | as reference the smallest sum on study (this includes the baseline sum |
| | if that is the smallest on study). In addition to the relative increase of |
| | 20%, the sum must also demonstrate an absolute increase of at least 5 |
| | mm. (Note: the appearance of one or more new lesions is also |
| | considered progression). |
| Stable | Neither sufficient shrinkage to qualify for PR nor sufficient increase to |
| Disease (SD) | qualify for PD, taking as reference the smallest sum diameters while on |
| | study |

TABLE 3
Evaluation of Non-Target Lesions

| Response | |
|---|---|
| Classification | Criteria |
| Complete | Disappearance of all nontarget lesions and normalization of tumor |
| Response (CR) | marker level. All lymph nodes must be nonpathological in size (<10 |
| | mm short axis). |
| Incomplete | Persistence of one or more nontarget lesion(s) and/or maintenance of |
| Response/Stable | tumor marker level above the normal limits. |
| Disease (SD) | |
| Progressive | Unequivocal progression (see comments below) of existing nontarget |
| Disease (PD) | lesions. (Note: the appearance of one or more new lesions is also |
| | considered progression). |

Table 4 provides a summary of the overall response status calculation at each time point for patients who have measurable disease at baseline. If patients have non-measurable (therefore non target) disease only, Table 5 is to be used. The best overall response (Table 6) is determined once all the data for the patient is known.

TABLE 4
Evaluation of Overall Response

| Target lesions | Non-target lesions | New lesions | Overall response |
|---|---|---|---|
| CR | CR | No | CR |
| CR | Non-CR/non-PD | No | PR |
| CR | Not evaluated | No | PR |
| PR | Non-PD or | No | PR |
| | not all evaluated | | |
| SD | Non-PD or | No | SD |
| | not all evaluated | | |
| Not all evaluated | Non-PD | No | NE (Not evaluated) |
| PD | Any | Yes or No | PD |
| Any | PD | Yes or No | PD |
| Any | Any | Yes | PD |

TABLE 5
Evaluation of Overall Response Status for Non-Measurable Disease

| Non-target lesions | New lesions | Overall response |
|---|---|---|
| CR | No | CR |
| Non-CR/non-PD | No | Non-CR/non-PD |
| Not all evaluated | No | NE (Not evaluated) |
| Unequivocal PD | Yes or No | PD |
| Any | Yes | PD |

TABLE 6
Evaluation of Best Overall Response

| | Overall | |
|---|---|---|
| Overall | response | |
| response First | Subsequent | |
| time point | time point | Best overall response |
| CR | CR | CR |
| CR | PR | SD, PD or PR |
| CR | SD | SD provided minimum criteria for SD |
| | | duration met; otherwise, PD |
| CR | PD | SD provided minimum criteria for SD |
| | | duration met; otherwise, PD |
| CR | NE | SD provided minimum criteria for SD |
| | | duration met; otherwise not evaluated |
| | | (NE) |
| PR | CR | PR |
| PR | PR | PR |
| PR | SD | SD |
| PR | PD | SD provided minimum criteria for SD |
| | | duration met, otherwise, PD |
| PR | NE | SD provided minimum criteria for SD |
| | | duration met, otherwise NE |
| NE | NE | NE |

When SD is believed to be best response, it must also meet a minimum time from baseline, defined as 42 days. If the minimum time is not met when SD is otherwise the best time point response, the patient's best response depends on the subsequent assessments. For example, a patient who has SD at first assessment, PD at second and does not meet minimum duration for SD, will have a best response of PD. The same patient lost to follow-up after the first SD assessment would be considered unevaluable. Complete or partial responses may be claimed only if the criteria for each are met at a subsequent time point (e.g., four weeks later).

Progression free survival is defined as the time (i.e., the time from the date of amcenestrant treatment start) until the occurrence of objective PD (i.e., tumor progression), according to RECIST 1.1 definitions (Response Evaluation Criteria in Solid Tumors, version 1.1), or death due to any cause, whichever occurs first.

Objective response rate is defined as the proportion of patients who have a confirmed complete response (CR) or partial response (PR), as best overall response (BOR) derived from overall response determined as per RECIST 1.1, from the date of amcenestrant treatment start to the date of end of treatment. In a clinical trial, such determination of BOR is made by the ICR.

Overall survival is defined as the time (i.e., the time from the date of amcenestrant treatment start) to the date of death (due to any cause).

Disease control rate is defined as the proportion of patients who have a confirmed CR, PR, stable disease (SD), or non-CR/non-PD as BOR as per RECIST 1.1 from the date of amcenestrant treatment start to the date of end of treatment.

Clinical benefit rate is defined as the proportion of patients who have a confirmed CR, PR, SD, or Non-CR/Non-PD for at least 24 weeks as per RECIST 1.1, from the date of amcenestrant treatment start to the date of end of treatment.

In some embodiments, PFS is assessed in patients based on their baseline ESRJ status (mutated or wild type). ESRJ status may be determined by, e.g., using multiplex droplet digital polymerase chain reaction (ddPCR) after extraction of plasma circulating DNA.

In some embodiments, the present treatment methods result in survival in the patient.

In some embodiments, the present treatment methods result in a median progression-free survival of 3.6 months (95% CI: 2.0 to 3.9).

In some embodiments, the present treatment methods result in a progression-free survival rate at six months of 35.5% (95% CI: 27.2 to 43.9).

In some embodiments, the present treatment methods result in a progression-free survival rate at 12 months of 20.4% (95% CI: 13.4 to 28.4).

In some embodiments, the present treatment methods result in complete response (CR), partial response (PR) or stable disease (SD) in the patient.

In some embodiments, the present treatment methods result in an Objective Response Rate of 11.9% (95% CI: 7.1 to 18.4).

In some embodiments, the present treatment methods result in a Clinical Benefit Rate of 27.3% (95% CI: 20.2 to 35.3).

In some embodiments, the present treatment methods result in a Disease Control Rate of 54.5% (95% CI: 46.0 to 62.9).

In further embodiments, the therapy achieves this result in patients with ER+/HER2− locally advanced/metastatic breast cancer with prior exposure to hormonal therapy.

### V. Articles of Manufacture and Kits

The present disclosure also provides articles of manufacture, e.g., kits, comprising one or more dosages of amcenestrant or a pharmaceutically acceptable salt thereof, and instructions for patients (e.g., for treatment in accordance with a method described herein). Amcenestrant tablets or capsules may be blistered and then carded. In some embodiments, each amcenestrant capsule or tablet contains 100 mg of amcenestrant.

### VI. Exemplary Embodiments

Non-limiting, exemplar embodiments of the present disclosure are further described below.

Embodiment 1: A method for reducing disease progression or death, including risk thereof, as compared to treatment with a therapy selected from fulvestrant, an aromatase inhibitor, and a selective estrogen receptor modulator, in a patient in need thereof who has ER+/HER2− advanced/metastatic breast cancer with mutated estrogen receptor 1 (ESR1), the method comprising administering to the patient amcenestrant or a pharmaceutically acceptable salt thereof.

Embodiment 2: The method of Embodiment 1, providing a reduction of 10.0% in risk of disease progression or death.

Embodiment 3: The method of Embodiment 1 or 2, wherein the aromatase inhibitor is exemestane, letrozole, or anastrozole.

Embodiment 4: The method of Embodiment 1 or 2, wherein the selective estrogen receptor modulator is tamoxifen.

Embodiment 5: The method of any one of Embodiments 1-4, wherein the method increases the progression-free survival (PFS) of the patient.

Embodiment 6: The method of any one of Embodiments 1-5, wherein the method results in a median PFS of about 3.7 months.

Embodiment 7: The method of any one of Embodiments 1-6, wherein amcenestrant or a pharmaceutically acceptable salt thereof is administered to the patient orally at a dose of 400 mg daily, optionally once daily.

Embodiment 8: The method of any one of Embodiments 1-7, wherein amcenestrant or a pharmaceutically acceptable salt thereof is provided as a capsule or a tablet, optionally comprising 100 mg of amcenestrant per capsule.

Embodiment 9: The method of any one of Embodiments 1-8, wherein amcenestrant or a pharmaceutically acceptable salt thereof is administered to the patient in the morning regardless of food status

Embodiment 10: The method of any one of Embodiments 1-9, wherein the breast cancer is an advanced breast cancer.

Embodiment 11: The method of Embodiment 10, wherein the advanced breast cancer is a locally advanced cancer which is not amenable to radiation therapy or surgery in a curative intent.

Embodiment 12: The method of any of Embodiments 1-9, wherein the breast cancer is metastatic.

Embodiment 13: The method of any one of Embodiments 1-12, wherein the patient is a pre- or post-menopausal woman, or a man.

Embodiment 14: The method of Embodiment 13, wherein the patient is a post-menopausal woman.

Embodiment 15: The method of any one of Embodiments 1-14, wherein the patient is resistant to endocrine therapy.

Embodiment 16: The method of any one of Embodiments 1-15, wherein the patient has been previously treated with at least one line, optionally one or two lines, of endocrine therapy for advanced breast cancer, optionally wherein the patient's breast cancer progressed during or after treatment with the previous endocrine therapy.

Embodiment 17: The method of Embodiment 16, wherein the patient has been previously treated with adjuvant endocrine therapy and relapsed after the first two years of the adjuvant endocrine therapy or within 12 months of completing the adjuvant endocrine therapy.

Embodiment 18: The method of Embodiment 17, wherein the previous adjuvant endocrine therapy is selected from treatment with tamoxifen, fulvestrant, or an aromatase inhibitor, optionally wherein the aromatase inhibitor is exemestane, letrozole, or anastrozole.

Embodiment 19: The method of any one of Embodiments 1-18, wherein the patient has been previously treated with chemotherapy or targeted therapy.

Embodiment 20: The method of Embodiment 19, wherein the patient has been previously treated with no more than one chemotherapy or one targeted therapy for advanced or metastatic disease.

Embodiment 21: The method of any one of Embodiments 1-20, wherein the patient has been previously treated with a CDK4/6 inhibitor.

Embodiment 22: The method of any one of Embodiments 1-21, wherein the patient has not been previously treated with an mTOR inhibitor and/or with a SERD other than fulvestrant.

Embodiment 23: A method of treating ER+/HER2− advanced/metastatic breast cancer with mutated estrogen receptor 1 (ESR1) in a patient in need thereof, the method comprising administering to the patient amcenestrant, or a pharmaceutically acceptable salt thereof.

Embodiment 24: The method of Embodiment 23, wherein treating is a reduction of 10.0% in risk of disease progression or death as compared to treatment with a therapy selected from fulvestrant, an aromatase inhibitor, and a selective estrogen receptor modulator/

Embodiment 25: The method of Embodiment 24, wherein the aromatase inhibitor is exemestane, letrozole, or anastrozole.

Embodiment 26: The method of Embodiment 24, wherein the selective estrogen receptor modulator is tamoxifen.

Embodiment 27: The method of any one of Embodiments 23-26, wherein the method increases the progression-free survival (PFS) of the patient.

Embodiment 28: The method of any one of Embodiments 23-27, wherein the method results in a median PFS of about 3.7 months.

Embodiment 29: The method of any one of Embodiments 23-28, wherein amcenestrant or a pharmaceutically acceptable salt thereof is administered to the patient orally at a dose of 400 mg daily, optionally once daily.

Embodiment 30: The method of any one of Embodiments 23-29, wherein amcenestrant or a pharmaceutically acceptable salt thereof is provided as a capsule or a tablet, optionally comprising 100 mg of amcenestrant per capsule.

Embodiment 31: The method of any one of Embodiments 23-30, wherein amcenestrant or a pharmaceutically acceptable salt thereof is administered to the patient in the morning regardless of food status.

Embodiment 32: The method of any one of Embodiments 23-31, wherein the breast cancer is an advanced breast cancer.

Embodiment 33: The method of Embodiment 32, wherein the advanced breast cancer is a locally advanced cancer which is not amenable to radiation therapy or surgery in a curative intent.

Embodiment 34: The method of any of Embodiments 23-31, wherein the breast cancer is metastatic.

Embodiment 35: The method of any one of Embodiments 23-34, wherein the patient is a pre- or post-menopausal woman, or a man.

Embodiment 36: The method of Embodiment 35, wherein the patient is a post-menopausal woman.

Embodiment 37: The method of any one of Embodiments 23-36, wherein the patient is resistant to endocrine therapy.

Embodiment 38: The method of any one of Embodiments 23-37, wherein the patient has been previously treated with at least one line, optionally one or two lines, of endocrine therapy for advanced breast cancer, optionally wherein the patient's breast cancer progressed during or after treatment with the previous endocrine therapy.

Embodiment 39: The method of Embodiment 38, wherein the patient has been previously treated with adjuvant endocrine therapy and relapsed after the first two years of the adjuvant endocrine therapy or within 12 months of completing the adjuvant endocrine therapy.

Embodiment 40: The method of Embodiment 39, wherein the previous adjuvant endocrine therapy is selected from treatment with tamoxifen, fulvestrant, or an aromatase inhibitor, optionally wherein the aromatase inhibitor is exemestane, letrozole, or anastrozole.

Embodiment 41: The method of any one of Embodiments 23-40, wherein the patient has been previously treated with chemotherapy or targeted therapy.

Embodiment 42: The method of Embodiment 41, wherein the patient has been previously treated with no more than one chemotherapy or one targeted therapy for advanced or metastatic disease.

Embodiment 43: The method of any one of Embodiments 23-42, wherein the patient has been previously treated with a CDK4/6 inhibitor.

Embodiment 44: The method of any one of Embodiments 23-43, wherein the patient has not been previously treated with an mTOR inhibitor and/or with a SERD other than fulvestrant.

Embodiment 45: An article of manufacture or kit, comprising amcenestrant and instructions for use for treating ER+/HER2− breast cancer according to the method of Embodiments 1-44.

Embodiment 46: Amcenestrant or a pharmaceutically acceptable salt thereof for use in treating breast cancer in the method of any one of Embodiments 1-44.

Embodiment 47: Use of amcenestrant or a pharmaceutically acceptable salt thereof in the manufacture of a medicament for treating breasting cancer in the method of any one of Embodiments 1-44.

Embodiment 48: The method of any only of Embodiments 1-44, wherein the patient in need thereof experiences no clinically significant bradycardia, QTc prolongation, or visual disturbances.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of oncology, medicine, medicinal and pharmaceutical chemistry, and cell biology described herein are those well-known and commonly used in the art. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Any compound disclosed herein can be used in any of the treatment method here, wherein the individual to be treated is as defined anywhere herein. Further, headers herein are created for ease of organization and are not intended to limit the scope of the claimed invention in any manner.

### LIST OF ABBREVIATIONS

AE: adverse event
AESI: adverse event of special interest
AI: aromatase inhibitor
ALT: alanine aminotransferase
ANC: absolute neutrophil count
AST: aspartate aminotransferase
BOR: best overall response
CBR: clinical benefit rate
cfDNA: cell-free deoxyribonucleic acid
CI: confidence interval
COD: cut-off date
CR: complete response
CT: computed tomography
CYP: cytochrome P450
DCR: disease control rate
DNA: deoxyribonucleic acid
ECOG: Eastern Cooperative Oncology Group
EOT: end of treatment
ER: estrogen receptor
ESR1: estrogen receptor 1 gene
FSH: follicle-stimulating hormone
GnRH: gonadotropin-releasing hormone
HER2: human epidermal growth factor receptor 2
HR: hazard ratio
IB: Investigator's Brochure
ICH: International Council for Harmonisation
ICR: independent central review
IHC: immunohistochemistry
IM: intramuscular
IMP: investigational medicinal product
INR: international normalized ratio
IRB: Institutional Review Board
IRT: Interactive Response Technology
ITT: intent-to-treat
LFT: liver function test
MedDRA: Medical Dictionary for Regulatory Activities
NCI-CTCAE: National Cancer Institute Common Terminology Criteria for Adverse Events
ORR: objective response rate
OS: overall survival
PCEM: Physician Choice Endocrine Monotherapy
PD: progressive disease
PFS: progression-free survival
PgR: progesterone receptor
PO: per os
PR: partial response
QD: once daily
RECIST: Response Evaluation Criteria in Solid Tumors
RNA: ribonucleic acid
SAE: serious adverse event
SD: stable disease
SERD: selective estrogen receptor degrader
SERM: selective estrogen receptor modulator
eTEAE: treatment-emergent adverse event
UGT: Uridine 5′-diphospho-glucuronosyltransferase
ULN: upper limit of normal

### EXAMPLES

AMEERA-3: Open Label Randomized Phase 2 Trial of Amcenestrant Versus Endocrine Monotherapy as Per Physician's Choice in Patients with Estrogen Receptor (ER)-Positive, HER2-Negative Locally Advanced or Metastatic Breast Cancer with Prior Exposure to Hormonal Therapies.

### Example 1: Clinical Trial Protocol

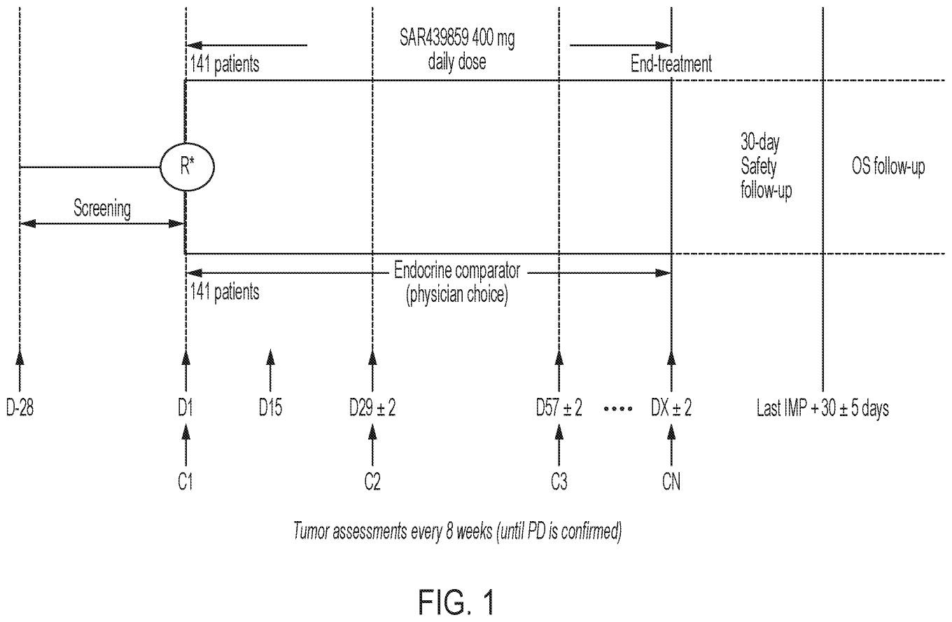

This Example describes the clinical trial protocol used for the study described below. This study was an open label randomized Phase 2 trial of amcenestrant monotherapy versus endocrine monotherapy as per physician's choice in patients with estrogen receptor (ER)-positive, HER2-negative locally advanced or metastatic breast cancer with prior exposure to hormonal therapies (FIG. 1). For the purpose of this study, a cycle was defined as a four-week period.

### Study Objectives

The primary objective of the study was to determine whether amcenestrant 400 mg via oral administration improves progression-free survival (PFS) when compared with an endocrine monotherapy of the choice of the physician, in patients with metastatic or locally advanced breast cancer.

A key secondary objective was to compare the overall survival between treatment arms.

Other secondary objectives were to evaluate in the two treatment arms:
i. the objective response rate,
ii. the disease control rate,
iii. the clinical benefit rate,
iv. the progression-free survival (PFS) according to the estrogen receptor 1 gene (ESR1) mutation status, and
v. the overall safety profile, in the two treatment arms.

### Study Endpoints

The primary endpoint of the study was the measurement of PFS, defined as the time interval from the date of randomization to the date of first documented tumor progression as per Response Evaluation Criteria in Solid Tumors (RECIST 1.1) assessed by independent central review (ICR) or death (due to any cause), whichever comes first.

Secondary endpoints are defined as follows:
Overall survival: the time interval from the date of randomization to the date of documented death (due to any cause);
Objective response rate: the proportion of participants who have a confirmed complete response (CR) or partial response (PR), as best overall response (BOR) derived from overall response determined by ICR as per RECIST 1.1, from the date of randomization to the date of end of treatment;
Disease control rate: the proportion of participants who have a confirmed CR, PR, stable disease (SD), or Non-CR/Non-PD as BOR determined by ICR as per RECIST 1.1 from the date of randomization to the date of end of treatment;
Clinical benefit rate: the proportion of participants who have a confirmed CR, PR, SD, or Non-CR/Non-PD for at least 24 weeks determined by ICR as per RECIST 1.1, from the date of randomization to the date of end of treatment;
Progression-free survival as per ESR1 status: determined at Cycle 1 Day 1;
Overall safety profile: evaluated by adverse events/serious adverse events and laboratory abnormalities.

### Study Design

This is an international, prospective, open-label, Phase 2 randomized study. Men, postmenopausal women, and premenopausal women on a gonadotropin-releasing hormone analog with locally advanced or metastatic breast cancer were randomly (1:1) assigned to one of the following two treatment arms: amcenestrant or an endocrine monotherapy of the choice of the physician (FIG. 1).

The randomization was stratified according to the presence of visceral metastasis (defined by at least one liver or lung metastasis) (Yes or No), prior treatment with CDK4/6 inhibitors (Yes or No), and Eastern Cooperative Oncology Group status (0 or 1).

Overall, 290 patients were randomly assigned to study intervention with a balanced randomization ratio in the global part of the study. The number of patients naïve to CDK4/6 inhibitors was limited to 20% of the overall sample size.

Patients were treated with either amcenestrant or a single endocrine therapy of choice of the physician, depending on the randomization allocation. The potential control treatment was selected in accordance with the Investigator's best clinical judgment before randomization.

### Dosing Regimen and Formulation

The amcenestrant arm was treated as follows:
Formulation: 100 mg capsules
Route(s) of administration: PO
Dose regimen: 400 mg (four capsules) QD, given in the morning, regardless of food status. Capsules were taken approximately at the same time every day (±3 hours). A cycle is artificially defined as a 4-week period.

Only one of the following single agent control treatments was allowed per patient randomized to the control arm. The treatment was selected before randomization in accordance with the Investigator's best clinical judgment, and treatment was administered as follows, with a cycle being defined as a 4-week period:
Fulvestrant (Faslodex®):
   Formulation: 50 mg/mL injection for IM administration
   Route(s) of administration: IM
   Dose regimen: 500 mg IM as two 250 mg (5 mL) injections, one injection in each buttock (gluteal area), on Cycle 1 Days 1 and 15, and at Day 1 of each 28 day cycle thereafter
AIs:
   Formulation: in accordance with the approved label
   Route of administration: PO
   Dose Regimen:
      Anastrozole: 1 mg QD, to be taken approximately at the same time every day, regardless of food status;
      Letrozole: 2.5 mg QD, to be taken approximately at the same time every day, regardless of food status; or
      Exemestane: 25 mg QD to be taken approximately at the same time every day after a meal.
Selective estrogen receptor modulator:
   Tamoxifen:
      Formulation: in accordance with the approved label
      Route of administration: PO
      Dose regimen: 20 mg to be taken once daily or 10 mg twice a day, approximately at the same time every day, regardless of food status.

Study duration for each individual patient included:
a period to assess eligibility (screening period) of up to four weeks (28 days) before randomization,
a treatment period of 28 days of study treatment per cycle, and
an end of treatment (EOT) visit at least 30 days (or until the patient receives another anticancer therapy, whichever is earlier) following the last administration of amcenestrant.

Study treatment was continued until precluded by unacceptable toxicity, disease progression, upon patient's request to stop treatment, or upon Investigator decision, whichever occurs first. Patients continue treatment after the last cut off date (COD). Patients who discontinue the study treatment without documented PD were followed every eight weeks until PD is documented or primary analysis COD, whichever occurred first.

### Study Population

Patients were eligible to be included in the study only if all of the following criteria applied:
I 01: Patient must be 18 years of age (inclusive) or older, at the time of signing the informed consent or country's legal age of majority if the legal age is more than 18 years;
I 02: Patients with histological or cytological proven diagnosis of adenocarcinoma of the breast.
I 03: Patients with evidence of either locally advanced disease not amenable to radiation therapy or surgery in a curative intent, and/or metastatic disease.
I 04: Documentation of ER positive (>1% positive stained cells) based on most recent tumor cell staining by immunohistochemistry (IHC) assay consistent with local standards (Note that if the primary tumor is ER positive and any further metastatic lesion is ER negative, the patient cannot be selected for inclusion).
I 05: Documentation of HER2 non over expressing based on most recent tumor sample by IHC (0, 1+), or in situ hybridization-negative based on single-probe average HER2 copy number <6.0 signals/cell or dual-probe HER2/centromeric probe for chromosome 17 (CEP17) ratio <2 with an average HER2 copy number <6.0 signals/cell as per American Society of Clinical Oncology guidelines (28) (Note that if the primary tumor was HER2 negative and any further metastatic lesion was HER2 positive, the patient could not be selected for inclusion).
I 06: deleted.
I 07: Prior chemotherapy (including antibody drug conjugates) or targeted therapy was allowed: patients must have received no more than one prior chemotherapeutic or one targeted therapy regimen for advanced/metastatic disease. For patients for whom CDK4/6 inhibitors were available (i.e., approved in their region and can be reimbursed), prior treatment with a CDK4/6 inhibitor in combination with fulvestrant or an AI was mandatory (Note: The number of patients naïve to CDK4/6 inhibitors should be limited to 20% of the overall sample size).
I 08: Patients must have received at least 6 months of a continuous prior endocrine therapy for advanced breast cancer and have progressed while on endocrine therapy (in single agent or in combination). The number of prior hormonal lines was limited to two. Patients with a relapse while on adjuvant endocrine therapy but after the first 2 years, or with a relapse within 12 months of completing adjuvant endocrine therapy were also eligible.
I 09: Male or Female.
   A) Postmenopausal women, as defined by one of the following:
      i) Women ≥60 years of age
      ii) Women <60 years of age:
         With spontaneous cessation of menses >12 months prior to randomization in the absence of chemotherapy, tamoxifen, and toremifene.
         Or with cessation of menses of duration ≤12 months or secondary to hysterectomy AND have follicle stimulating hormone (FSH) level in the postmenopausal range according to institutional standards (or >34.4 IU/L if institutional range is not available) prior to randomization.
         Or who have received hormonal replacement therapy but have discontinued this treatment AND have FSH level in the postmenopausal range according to institutional standards (or >34.4 IU/L if institutional range is not available) prior to randomization.
         Or with status post bilateral surgical oophorectomy.
         Or postbilateral ovarian ablation through pelvic radiotherapy.
   B) Pre/perimenopausal women, i.e., not meeting the criteria for being postmenopausal.
   C) Male patients.
   Note: Male with no prior bilateral orchiectomy and pre/perimenopausal women should be on a GnRH agonist for at least 4 weeks prior to randomization (to be continued during study treatment).
I 10: Capable of giving signed informed consent, which includes compliance with the requirements and restrictions listed in this protocol.
I 11: Patients must be considered clinically eligible by the Investigator to receive single agent endocrine therapy.

Patients were excluded from the study if any of the following criteria apply:
E 01: Eastern Cooperative Oncology Group performance status ≥2.
E 02: Significant concomitant illness, including psychiatric condition that, in the opinion of the Investigator or Sponsor, would adversely affect the patient's participation in the study.
E 03: Medical history or ongoing gastrointestinal disorders potentially affecting the absorption of oral IMP. Patients unable to swallow normally and to take capsules. Predictable poor compliance to oral treatment.
E 04: Patients not suitable for participation, whatever the reason, as judged by the Investigator, including medical or clinical conditions, or patients potentially at risk of noncompliance to the study procedures (ie, unwillingness and inability to comply with scheduled visits, drug administration plan, laboratory tests, other study procedures, and study restrictions).
E 05: Major surgery within 4 weeks prior to randomization.
E 06: Patient with any other cancer. Adequately treated basal cell or squamous cell skin cancer or in situ cervical cancer or any other cancer from which the patient has been disease free for >3 years are allowed.
E 07: Any medical conditions that are contraindicated to endocrine treatment of physician's choice (refer to approved label for details).
E 08: Patients with abnormal coagulation profiles or any history of coagulopathy within the 6 months prior to the first dose of IMP, including history of deep vein thrombosis or pulmonary embolism. However, patients with the following conditions will be allowed to participate:
   Patients with adequately treated catheter related venous thrombosis that occurred more than 1 month prior to the first dose of IMP.
   Patients being treated with an anticoagulant (e.g., warfarin or heparin) for a thrombotic event that occurred more than 6 months before enrollment, or for an otherwise stable and allowed medical condition (eg, well controlled atrial fibrillation), provided that dose and coagulation parameters (as defined by local standard of care) were stable for at least 1 month prior to the first dose of IMP.
E 09: Patients with a life expectancy <3 months.
E 10: deleted.
E 11: Patients with known brain metastases that were untreated, symptomatic or required therapy to control symptoms. Patients with brain metastases were eligible if they:
   Had completed treatment (whole brain radiotherapy, radiosurgery, or combination) at least 4 weeks prior to start of study treatment, and
   Had recovered from the effects of this treatment, and
   Were neurologically stable.
      Any corticosteroid use for brain metastases must have been discontinued without the subsequent appearance of symptoms for ≥2 weeks prior to first IMP.
E 12: No improvement of any prior treatment related adverse reaction to <Grade 2, except for alopecia according to the National Cancer Institute Common Terminology Criteria for Adverse Events (NCI CTCAE) v5.0.
E 13: Prior treatment with mammalian target of rapamycin inhibitors or any other SERD compound, except fulvestrant if stopped for at least three months before randomization.
E 14: Treatment with drugs that have the potential to inhibit UGT, including, but not limited, to atazanavir and probenecid, for less than two weeks before randomization or 5 elimination half-lives whichever is longer.
E 15: Treatment with strong CYP3A inducers within two weeks before randomization or 5 elimination half-lives whichever is longer.
E 16: Ongoing treatment with drugs that are sensitive substrate of OATP1B1/B3 (asunaprevir, atorvastatin, bosentan, danoprevir, fexofenadine, glyburide, nateglinide, pitavastatin, pravastatin, repaglinide, rosuvastatin, and simvastatin acid).
E 17: Treatment with anticancer agents (including investigational drugs) less than 3 weeks before randomization.
E 18: Inadequate hematological function including neutrophils <1.5×10⁹/L platelet count <100×10⁹/L.
E 19: Prothrombin time/international normalized ratio (INR)>1.5 times the upper limit of normal (ULN) or outside therapeutic range if receiving anticoagulation that would affect the prothrombin time/INR.
E 20: Inadequate renal function with serum creatinine ≥1.5×ULN or between 1.0 and 1.5×ULN with glomerular filtration rate <60 mL/min/1.73 m² as estimated using the abbreviated Modification of Diet in Renal Disease formula.
E 21: Liver function:
   Aspartate aminotransferase >3×ULN, or ALT>3×ULN.
   Total bilirubin >1.5×ULN.
   Note: In the presence of hepatic metastases, AST and ALT<5×ULN are acceptable.
E 22: Patients accommodated in an institution because of regulatory or legal order; prisoners or patients who were legally institutionalized.
E 23: Patients dependent on the Sponsor or Investigator (in conjunction with Section 1.61 of the International Council for Harmonisation-Good Clinical Practice Ordinance E6).
E 24: Employees of the clinical study site or other individuals directly involved in the conduct of the study, or immediate family members of such individuals.
E 25: Sensitivity to any of the study interventions, or components thereof, or drug or other allergy that, in the opinion of the Investigator, contraindicated participation in the study.

### Concomitant Therapy

Patients were asked to abstain from taking prescription or nonprescription drugs (including vitamins and dietary or herbal supplements) within seven days (or 14 days if the drug is a potential enzyme inducer) or five half-lives (whichever is longer) before the start of treatment until completion of the follow-up visit, unless, in the opinion of the Investigator and Sponsor, the medication would not interfere with the study.

Bisphosphonates and receptor activator of nuclear factor kappa B ligand inhibitors were allowed.

Special caution was taken regarding the following therapies:
No additional investigational or commercial anticancer agents such as chemotherapy, immunotherapy, targeted therapy, biological response modifiers, or endocrine therapy other than amcenestrant or the selected control were permitted during the active treatment phase. In general, any drugs containing “for the treatment of breast cancer” on the product label were not permitted on study.
Palliative radiotherapy was given for control of pain for palliative intents. The irradiated area was as small as possible and, never involved more than 20% of the bone marrow in any given three week period. In all such cases, the possibility of tumor progression was ruled out by physical and radiological assessments of the tumor. If the only evaluable lesions were to be irradiated, the patient would stop the study treatment. The irradiated area could not be used as a parameter for response assessment.
Drugs which are sensitive substrates of CYP3A, CYP2B6, CYP2Cs, and/or UGT should be closely monitored since there may be loss of efficacy of these drugs by concomitant use of amcenestrant due to a potential induction effect of amcenestrant.

The following therapies/medications were prohibited throughout the active treatment phase for the amcenestrant treatment arm:
Drugs that are strong inducers of CYP3A, since they may decrease amcenestrant exposure.
Herbal medications and food supplements including St John's Wort and genistein during treatment period, since they could decrease amcenestrant exposure.
Drugs that are sensitive substrates of OATP1B1/1B3 including asunaprevir, atorvastatin, bosentan, danoprevir, fexofenadine, glyburide, nateglinide, pitavastatin, pravastatin, repaglinide, rosuvastatin, and simvastatin acid, since amcenestrant is a potential inhibitor and may decrease their elimination.
Drugs that have UGT inhibition potential and are contraindicated with UGT substrates, including, but not limited to, atazanavir and probenecid, since amcenestrant is substrate of UGT1A1 and UGT1A4.

### Study Assessments

Cycle 1 Day 1 refers to the day the participant received the initial dose of study treatment which was a single administration of amcenestrant, or control selected by the physician.

Cycle 1 Day 15 corresponds for participants randomized in the control arm and treated by fulvestrant to the second fulvestrant injection.

A cycle duration was 28 days. Day 1 of Cycle 1 refers to the day the participant received the first study treatment administration. Day 1 of each subsequent cycle corresponds to the visit performed on Day 29±2 days of previous cycle.

The primary and secondary efficacy endpoints were assessed by the RECIST 1.1 criteria. Tumor assessments were reviewed centrally by ICR and supported by investigator assessment.

Concerning monitoring of adverse events, mention may be made of adverse events of special interest (AESI), who correspond to an adverse event (serious or nonserious) of scientific and medical concern specific to the Sponsor's product or program, for which ongoing monitoring and immediate notification by the Investigator to the Sponsor is required. Such events may require further investigation in order to characterize and understand them. For example: pregnancy of a participant or of a female partner of a male participant entered in a study with IMP, symptomatic overdose with IMP, increase in ALT of a grade equal to or greater than 3, or photosensitivity.

### Populations for Analyses

The analysis populations were defined as follows:
Enrolled population: all patients who signed the informed consent form.
Intent-to-treat (ITT) population: all patients from the enrolled population and for whom there was a confirmation of successful allocation of a randomization number by Interactive Response Technology (IRT). Patients were analyzed according to the treatment arm assigned at randomization.
Safety population: all patients randomly assigned to study intervention and who took at least one dose of study intervention. Patients were analyzed according to the treatment arm they actually received.

### Efficacy Analyses

All efficacy analyses for the global part were performed on the intent-to-treat (ITT) population unless stated otherwise. All primary and secondary efficacy endpoints based on radiological assessments of tumor burden (i.e., PFS, BOR, ORR, disease control rate [DCR] and clinical benefit rate [CBR]) were derived using the ICR tumor assessment. Analysis based on local radiologist's/Investigator's assessment was considered as supportive analyses. A summary of efficacy analyses performed is provided in Table 7.

TABLE 7
Summary of Efficacy Analyses

| Endpoint | Statistical Analysis Methods |
|---|---|
| PFS | Stratified logrank for statistical testing. |
| | Stratified Cox proportional hazard model for HR. |
| | Kaplan-Meier method for probabilities of being event free at |
| | different time points. |
| OS | Stratified logrank for statistical testing. |
| | Stratified Cox proportional hazard model for HR. |
| | Kaplan-Meier method for probabilities of being event free at |
| | different time points. |
| ORR, DCR, | No statistical testing was performed. |
| CBR | Descriptive statistics by treatment arm and Clopper-Pearson method |
| | for CI calculation. |
| PFS by ESR1 | Stratified Cox proportional hazard model for HR. |
| mutation status | Kaplan-Meier method for probabilities of being event free at |
| | different time points. |

CBR = clinical benefit rate; CI = confidence interval; DCR = disease control rate; ESR1 = estrogen receptor 1 gene; HR = hazard ratio; OS = overall survival; ORR = objective response rate; PFS = progression-free survival.

Primary efficacy analysis consisted of PFS comparison (based on ICR) between the amcenestrant arm and the control arm through a logrank test procedure stratified by the stratification factors. A one sided Type I error rate of 2.5% was used for statistical testing.

The primary analysis of PFS was based on the following censoring rules:
If progression and death were not observed before the COD for final PFS, PFS was censored at the date of the last valid disease assessment with no evidence of a disease progression prior to the initiation of a further anticancer therapy (if any).
A patient without an event (death or disease progression) and without any valid postbaseline disease assessments was censored at the day of randomization (Day 1).
A patient with an event documented after two or more non-evaluable tumor assessments was censored at the date of the last evaluable tumor assessment documenting no progression prior to the initiation of a further anticancer therapy.

The COD for final PFS analysis is the date when approximately 201 PFS events assessed by ICR are observed or when all patients from the global cohort have been followed-up for at least 10 months (or discontinued treatment), whichever is earlier.

The HR estimates and corresponding 95% two-sided CIs were provided using the Cox proportional hazard model stratified by the same stratification factors as those used for the logrank test described above. Progression-free survival for the two treatment arms was summarized using Kaplan-Meier methods and displayed graphically. In addition to the median PFS, the 25^{th}, 50^{th} and 75^{th} percentiles event time and associated 95% CI were provided, along with probabilities of being progression free at different time points.

In the case of overall survival (OS), in the absence of observation of death, survival time was censored to the last date the patient is known to be alive. The HR estimates and corresponding 95% two-sided CI was provided using the Cox proportional hazard model stratified by the same stratification factors as those used for the PFS analysis described above. Overall survival for the two treatment arms was summarized using Kaplan Meier methods and displayed graphically. The median, 25^{th}, 50^{th} and 75^{th} percentiles event time and associated 95% CI were provided. The survival probability and its 95% CI was estimated using the Kaplan-Meier method and a log-log approach based on a normal approximation following the Greenwood's formula. In order to ensure a strong control of the overall Type I error rate at a one-sided 2.5%, a hierarchical testing strategy was defined. In other words, comparison between arms on the OS was to be performed only if the primary analysis of the PFS was statistically significant. In case of statistically significant PFS, OS was to be compared between treatment arms through a logrank test procedure stratified by the stratification factors as entered in the IRT. Otherwise, descriptive statistics of OS was to be provided at the time of final PFS analysis. The COD for final OS analysis was set to the date when approximately 196 death events would have been observed (approximately 70% of the patients have died).

The objective response rate (ORR) on each randomized treatment arm was estimated by dividing the number of patients with objective response (confirmed CR or PR as BOR, according to RECIST 1.1) by the number of patients from the analysis population of the respective treatment arm. In addition, 95% two sided CIs were computed using the Clopper Pearson method.

The disease control rate (DCR) on each randomized treatment arm was estimated by dividing the number of patients with disease control (confirmed CR or PR, SD, or Non-CR/Non-PD as BOR, according to RECIST 1.1) by the number of patients from the analysis population of the respective treatment arm. In addition, 95% two-sided CIs were computed using the Clopper Pearson method.

The clinical benefit rate (CBR) on each randomized treatment arm was estimated by dividing the number of patients with clinical benefit (confirmed CR or PR as BOR, SD or Non-CR/Non-PD lasting at least 24 weeks, according to RECIST 1.1) by the number of patients from the analysis population of the respective treatment arm. In addition, 95% two-sided CIs were computed using the Clopper Pearson method.

### Example 2: Clinical Trial Results

The results presented herein stem from a patient database cut-off date of 15 Feb. 2022.

### Patient Population

A total of 290 patients were randomized in the study, with 143 patients randomized in the amcenestrant arm and 147 in the control arm.

Patients in the control arm were treated with the following endocrine therapies:
SERD (fulvestrant): 132 patients (89.8%);
Aromatase inhibitors: 10 patients (6.8%), with 4 patients taking letrozole (2.7%) and 6 patients taking exemestane (4.1%);
SERM (tamoxifen): 5 patients (3.4%).

Hence, patients in the control arm were mostly treated with fulvestrant.

The main baseline characteristics of the patients were balanced between treatment arms and are described in Table 8.

TABLE 8
Main Demographic and Other Baseline Characteristics - ITT Population

| | | |
|---|---|---|
| PCEM | Amcenestrant | |
| (N = 147) | (N = 143) | All (N = 290) |

Demography

| | | | | | | |
|---|---|---|---|---|---|---|
| Age, median [range) | 60 | (28; 86) | 58 | (29; 84) | 59 | (28; 86) |
| ECOG PS 0, n (%) | 94 | (63.9) | 97 | (67.8) | 191 | (65.9) |
| Postmenopausal, n (%) | 128 | (87.7) | 117 | (81.8) | 245 | (84.8) |

Disease Characteristics

| | | | | | | |
|---|---|---|---|---|---|---|
| Measurable disease per ICR, n (%) | 125 | (85.0) | 129 | (90.2) | 254 | (87.6) |
| Visceral metastasis¹ per ICR, n (%) | 94 | (63.9) | 91 | (63.6) | 185 | (63.8) |
| Bone-only disease per ICR, n (%) | 12 | (8.2) | 9 | (6.3) | 21 | (7.2) |
| ER+, n (%) | 147 | (100) | 143 | (100) | 290 | (100) |
| PgR+, n (%) | 106 | (72.1) | 106 | (74.1) | 212 | (73.1) |
| HER2−, n (%) | 146 | (99.3)* | 143 | (100) | 289 | (99.7) |
| ESR1 mutated, n (%) | 55 | (39.3) | 65 | (46.4) | 120 | (42.9) |

Prior treatment in advanced setting

| | | | | | | |
|---|---|---|---|---|---|---|
| Number of prior lines, n (%) | | | | | | |
| 0 | 10 | (6.8) | 8 | (5.6) | 10 | (6.8) |
| 1 | 117 | (79.6) | 116 | (81.1) | 117 | (79.6) |
| 2 | 18 | (12.2) | 16 | (11.2) | 18 | (12.2) |
| ≥3 | 2 | (1.4) | 3 | (2.1) | 2 | (1.4) |
| Prior chemotherapy, n (%) | 19 | (12.9) | 14 | (9.8) | 33 | (11.4) |
| Prior hormonotherapy, n (%) | 137 | (93.2) | 134 | (93.7) | 271 | (93.4) |
| Prior targeted therapy², n (%) | 115 | (78.2) | 114 | (79.7) | 229 | (79.0) |

¹Defined as lung or liver lesions per ICR.
²All patients who received a prior targeted therapy received a CDK4/6i.
*Erroneous randomization of one HER2+ patient in the control arm due to result available post-randomization.

All patients were ER positive, and 73.1% of the patients were progesterone receptor positive (PgR). With the exception of one patient, all patients were HER2 negative.

Overall, the population is representative of the ER+, HER2− advanced/metastatic breast cancer patients who are resistant to endocrine therapy expected in the trial, with:
84.8% postmenopausal status;
63.8% visceral involvement per ICR;
42.9% ESR1-mutated;
79.6% of patients entering in second line treatment; and
79% prior treatment with CDK4/6i.

### Exposure

At the cut-off date, 20 patients remained on treatment in each study arm: 123 patients had discontinued treatment in the amcenestrant arm, and 127 patients had discontinued treatment in the control arm, due mainly to occurrence of PD.

The duration of IMP exposure was as follows:
Mean (standard deviation) duration: 26.3 weeks (23.7) in the amcenestrant arm and 25.4 weeks (23.5) in the PCEM arm.
Median duration: 16.0 weeks in the amcenestrant arm and 15.9 weeks in the PCEM arm.
Minimum to maximum exposure duration: 0 to 116 weeks in the amcenestrant arm and 2 to 102 weeks in the PCEM arm.

The mean (standard deviation) Relative Dose, defined as the ratio of the Total Cumulative Dose to the Total Planned Dose, was 97.7% (9.6) and 97.3% (8.4) in the PCEM and the amcenestrant arms, respectively.

### Efficacy Analysis—PFS

FIG. 2 shows the Kaplan-Meier graph of PFS assessed by ICR, in the ITT population. Results on PFS assessed per ICR (primary objective of the study) are as shown in Table 9.

TABLE 9
PFS Results per ICR - ITT Population

| | PCEM | | Amcenestrant | |
|---|---|---|---|---|
| | (N = 147) | | (N = 143) | |
| Number (%) of events | 95 | (64.6) | 100 | (69.9) |
| Median (95% CI) in months | 3.7 | (2.0 to 4.9) | 3.6 | (2.0 to 3.9) |
| Hazard ratio (95% CI) | | | 1.051 | (0.789 to 1.4) |

| | |
|---|---|
| One-sided p-value | 0.6437 |

At a median follow-up of 11.2 months, it hence appears that the risk of disease progression or death was numerically similar between the two treatment arms (HR=1.051 95% CI (0.789 to 1.4)). Median PFS (95% CI) were numerically similar between amcenestrant (3.6 months (2.0 to 3.9)) and PCEM (3.7 months (2.0 to 4.9)).

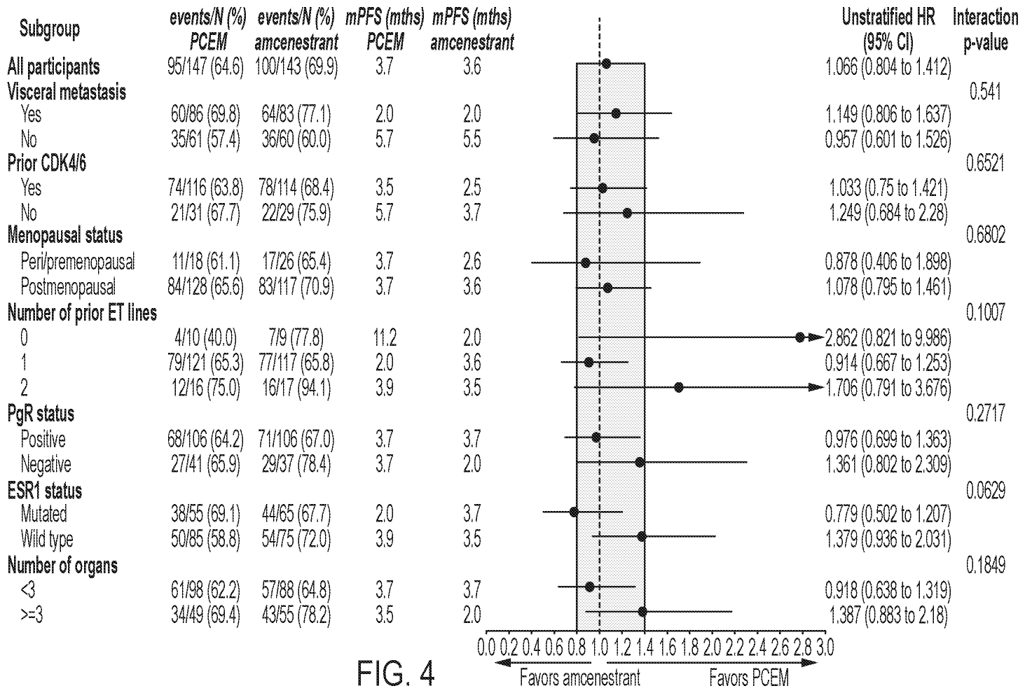

Findings on PFS show that the treatment effect was in general consistent across key sub-groups, defined by baseline characteristics (FIG. 4).

### ESR1-Wildtype Patients

Per ICR assessments, from the ESR1-wildtype participants in baseline, 54 (72.0%) and 50 (58.8%) participants had PFS events in the amcenestrant and PCEM arms, respectively. The stratified HR was 1.305 (95% CI: 0.88 to 1.937). The median PFS was 3.5 months (95% CI: 2.0 to 3.7) in the amcenestrant arm and 3.9 months (95% CI: 3.6 to 9.2) in the PCEM arm.

### ESR1-Mutated Patients

In ESR1-mutated patients at baseline, per ICR assessment it was observed that 44 (67.7%) and 38 (69.1%) participants had PFS events in the amcenestrant and PCEM arms, respectively (Table 10). The stratified HR was 0.9 (95% CI: 0.565 to 1.435), corresponding to a reduction of 10.0% in risk for disease progression or death with amcenestrant compared to PCEM arms. The median PFS was 3.7 months (95% CI: 1.9 to 7.2) in the amcenestrant arm versus 2.0 months (95% CI: 1.9 to 4.3) in the PCEM arm. So, overall the participants with ESR-1 mutation at baseline performed better with the amcenestrant therapy over the PCEM therapy.

TABLE 10
PFS - Primary Analysis Based on ICR Assessment -
ITT Population with ESR1 Mutation at Baseline

| | PCEM | Amcenestrant 400 mg |
|---|---|---|
| | (N = 55) | (N = 65) |
| Number (%) of events | 38 (69.1) | 44 (67.7) |
| Median PFS (months) | 2.0 | 3.7 |
| (95% CI) | (1.9 to 4.3) | (1.9 to 7.2) |
| Hazard ratio (95% CI) | | 0.9 (0.565 to |
| | | 1.435) |

ESR1-Y537S Mutated Patients Mutational profiling for 12 pathogenic ESR1 mutations was conducted at baseline (BL) and on treatment at Cycle 3, Day 1 (C3D1) by droplet digital polymerase chain reaction in plasma cell-free DNA. At BL, ESR1 mutations were present in 65/140 (46.4%) and 55/140 (39.3%) patients in the amcenestrant and PCEM arms, respectively, with a similar number and type of ESR1 mutations between arms. For patients harboring the ESR1-Y537S mutation (amcenestrant, N=27/140 [19.3%]; PCEM, N=26/141 [18.4%]), numerically longer PFS was observed with amcenestrant versus PCEM (median PFS 3.8 months versus 1.9 months; stratified HR=0.431 [95% CI. 0.205, 0.909]Error! Reference source not found.). In patients with ESR1 data at both BL and C3D1 (amcenestrant, N=90; PCEM, N=82), 20/44 patients with ESR1 mutations detected at BL were cleared on treatment with amcenestrant versus 2/29 for PCEM.

### Efficacy Analysis—OS

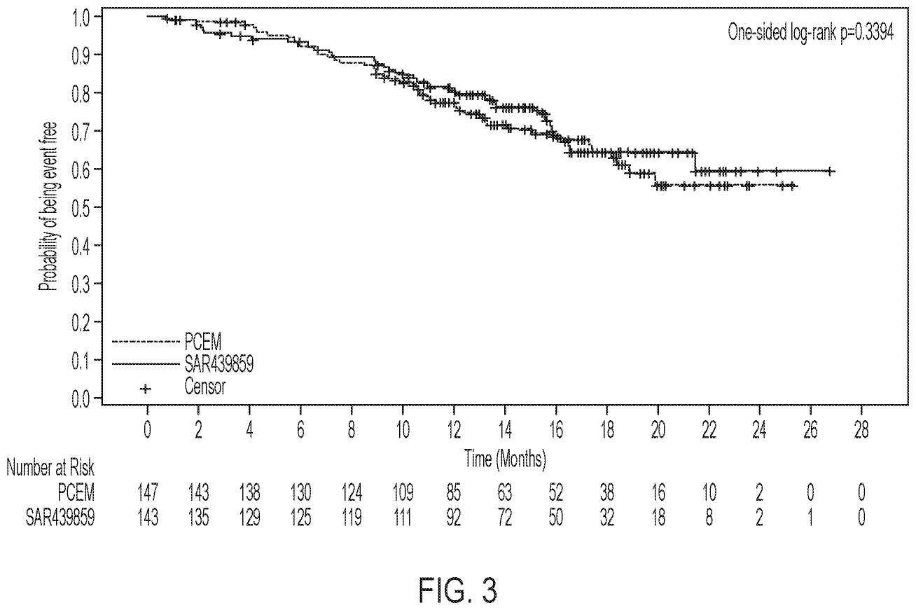

FIG. 3 shows the Kaplan-Meier graph of OS in the ITT population. Results of OS are as shown in Table 11.

TABLE 11
OS Results per ICR, ITT Population

| | PCEM | Amcenestrant |
|---|---|---|
| | (N = 147) | (N = 143) |
| Number (%) of events | 46 (31.3) | 40 (28.0) |
| Median (95% CI) | NC* (18.9 to | NC (21.5 to |
| | NC) | NC) |
| Hazard ratio (95% CI) | | 0.913 (0.595 to |
| | | 1.403) |
| One-sided p-value | | 0.3394 |

*Not Calculable

At a median follow-up of 16.2 months, a numerically similar survival trend was observed between the two treatment arms (HR=0.913 95% CI (0.595 to 1.403)), although OS assessment was not mature. The OS rate (95% CI) at 12 months was 80.4% (72.5 to 86.2) in the amcenestrant arm versus 77.4% (69.4 to 83.6) in the PCEM arm.

### Efficacy—Antitumor Activity

Tumor response-based secondary efficacy endpoints are described in Table 12.

TABLE 12
ICR-Assessed Antitumor Activity of Amcenestrant Compared with PCEM Groups

| | Population | | | |
|---|---|---|---|---|
| | ITT | | ITT with measurable disease | |
| | Treatment | | | |
| | Amcenestrant | PCEM | Amcenestrant | PCEM |
| n | 143 | 147 | 129 | 125 |
| Objective Response Rate^{a}, n (%) | 17 (11.9) | 13 (8.8) | 17 (13.2) | 13 (10.4) |
| [95% CI]^{b} | [7.1 to 18.4] | [4.8 to 14.6] | (7.9 to 20.3) | (5.7 to 17.1) |
| Clinical Benefit Rate^{c}, n (%) | 39 (27.3) | 43 (29.3) | 32 (24.8) | 34 (27.2) |
| [95% CI]^{b} | [20.2 to 35.3] | [22.0 to 37.3] | (17.6 to 33.2) | (19.6 to 35.9) |
| Disease Control Rate^{d}, n (%) | 78 (54.5) | 79 (53.7) | 69 (53.5) | 63 (50.4) |
| [95% CI]^{b} | (46.0 to 62.9) | (45.3 to 62.0) | [44.5 to 62.3] | [41.3 to 59.5] |

Abbreviations: CI, confidence interval; CR, complete response; ICR, independent central review; ITT, intent-to-treat; PD, progressive disease; PR, partial response; SD, stable disease; PCEM, Physician Choice Endocrine Monotherapy.
^{a}Confirmed CR or confirmed PR;
^{b}Estimated by Clopper-Pearson method;
^{c}CR, PR, or SD or non-CR/non-PD ≥24 weeks;
^{d}CR, PR, SD, or non-CR/non-PD.

### Safety

The overview of adverse events (TEAEs: Treatment Emergent Adverse Events; SAEs: Serious Adverse Events; AESI: Adverse Events of Special Interest) is presented in Table 13.

TABLE 13
Overview of Adverse Event Profile - TEAEs - Safety Population

| | PCEM | | Amcenestrant | |
|---|---|---|---|---|
| n (%) | (N = 147) | | (N = 143) | |
| Participants with any TEAE | 112 | (76.2) | 118 | (82.5) |
| Participants with any grade ≥3 TEAE | 23 | (15.6) | 31 | (21.7) |
| Participants with any grade 5 TEAE^{a} | 2 | (1.4) | 5 | (3.5) |
| Participants with any treatment emergent SAE | 15 | (10.2) | 23 | (16.1) |
| Participants with any TEAE leading to treatment | 2 | (1.4) | 5 | (3.5) |
| discontinuation | | | | |
| Participants with any treatment-related TEAE | 46 | (31.3) | 64 | (44.8) |
| Participants with any AESI^{b} | 3 | (2.0) | 1 | (0.7) |

^{a}Grade 5 TEAE occurring during the treatment period.
^{b}AESI include pregnancy, symptomatic overdose with IMP, increase in ALT Grade equal to or greater than 3, photosensitivity in some instances.

More frequent gastrointestinal disorders are observed in the amcenestrant treatment arm (44.8% in the amcenestrant arm versus 22.4% in the PCEM arm), mostly of Grade 1 or 2. Also, a higher rate of skin and subcutaneous tissue disorders is observed (18.9% in the amcenestrant arm versus 7.5% in the PCEM arm), again mostly of Grade 1 or 2.

Although a numerically greater incidence of adverse events is observed with amcenestrant compared to the PCEM arm, overall, the adverse event profile in amcenestrant treatment arm is consistent with earlier studies with the same product. Importantly, no bradycardia/QTc prolongation is observed in the amcenestrant treatment arm, as it was seen with some other SERDs in clinical development.

### CONCLUSION

AMEERA-3 study did not meet its primary objective on PFS per ICR, as amcenestrant did not prove to be statistically superior to PCEM. The observed risk of disease progression or death was numerically similar between treatment arms.

AMEERA-3 study showed that amcenestrant, when used at a 400 mg QD, is able to provide a numerically longer progression-free survival compared to endocrine therapy of physician's choice in ER+/HER2− advanced/metastatic breast cancer patients with ESR1-mutation. In patients with ER+/HER2− advanced/metastatic breast cancer harboring the ESR1-Y537S mutation, treatment with amcenestrant was associated with a numerically longer PFS compared to endocrine therapy of physician's choice.

The safety profile with amcenestrant is consistent with earlier study with that same product, with a numerically higher rate of TEAEs (all grades, grade ≥3 TEAEs, related TEAEs, and SAEs) observed with amcenestrant compared to PCEM and which is primarily driven by gastrointestinal disorders.

1. A method for reducing disease progression or death as compared to treatment with a therapy selected from fulvestrant, an aromatase inhibitor, and a selective estrogen receptor modulator, in a patient in need thereof, wherein the patient has ER+/HER2− advanced/metastatic breast cancer with mutated estrogen receptor 1 (ESR1), the method comprising administering to the patient amcenestrant or a pharmaceutically acceptable salt thereof.

2. A method of treating ER+/HER2− advanced/metastatic breast cancer with mutated estrogen receptor 1 (ESR1) in a patient in need thereof, the method comprising administering to the patient amcenestrant or a pharmaceutically acceptable salt thereof.

3. The method of claim 1, wherein the method results in a reduction of 10.0% in risk of disease progression or death as compared to treatment with a therapy selected from fulvestrant, an aromatase inhibitor, and a selective estrogen receptor modulator.

4. The method of claim 1, wherein the aromatase inhibitor is exemestane, letrozole, or anastrozole.

5. The method of claim 1, wherein the selective estrogen receptor modulator is tamoxifen.

6. The method of claim 1, wherein the method increases the progression-free survival (PFS) of the patient.

7. The method of claim 1, wherein the method results in a median PFS of about 3.7 months.

8. The method of claim 1, wherein amcenestrant or a pharmaceutically acceptable salt thereof is administered to the patient orally at a dose of 400 mg daily, optionally once daily.

9. The method of claim 1, wherein amcenestrant or a pharmaceutically acceptable salt thereof is provided as a capsule or a tablet, optionally comprising 100 mg of amcenestrant per capsule or tablet.

10. The method of claim 1, wherein amcenestrant or a pharmaceutically acceptable salt thereof is administered to the patient in the morning regardless of food status.

11. The method of claim 1, wherein the breast cancer is advanced breast cancer.

12. The method of claim 11, wherein the advanced breast cancer is locally advanced cancer that is not amenable to radiation therapy or surgery in a curative intent.

13. The method of claim 1, wherein the breast cancer is metastatic.

14. The method of claim 1, wherein the patient is a pre- or post-menopausal woman, or a man.

15. The method of claim 14, wherein the patient is a post-menopausal woman.

16. The method of claim 1, wherein the patient is resistant to endocrine therapy.

17. The method of claim 1, wherein the patient has been previously treated with at least one line, optionally one or two lines, of endocrine therapy for advanced breast cancer, optionally wherein the patient's breast cancer progressed during or after treatment with the previous endocrine therapy.

18. The method of claim 17, wherein the patient has been previously treated with adjuvant endocrine therapy and relapsed after the first two years of the adjuvant endocrine therapy or within 12 months of completing the adjuvant endocrine therapy.

19. The method of claim 18, wherein the previous adjuvant endocrine therapy is selected from treatment with tamoxifen, fulvestrant, or an aromatase inhibitor, optionally wherein the aromatase inhibitor is exemestane, letrozole, or anastrozole.

20. The method of claim 1, wherein the patient has been previously treated with chemotherapy or targeted therapy.

21. The method of claim 20, wherein the patient has been previously treated with no more than one chemotherapy or one targeted therapy for advanced or metastatic disease.

22. The method of claim 1, wherein the patient has been previously treated with a CDK4/6 inhibitor.

23. The method of claim 1, wherein the patient has not been previously treated with an mTOR inhibitor and/or with a SERD other than fulvestrant.

24. The method of claim 1, wherein the patient experiences no clinically significant bradycardia, QTc prolongation, or visual disturbances.

25. An article of manufacture or kit, comprising amcenestrant and instructions for use for treating ER+/HER2− breast cancer in the method of claim 1.

26. (canceled)

27. (canceled)
